(19) United States
(12) Patent Application Publication
(10) Pub. No.: US 2025/0333370 A1
(43) Pub. Date: Oct. 30, 2025

(54) METHOD FOR EXTRACTING 3,5-O-DICAFFEOYLQUINIC ACID FROM HUANGSHAN GONGJU AND ITS APPLICATION IN DRUGS FOR PROTECTING AGAINST ALCOHOLIC LIVER DAMAGE

(71) Applicant: Hefei University of Technology, Hefei City (CN)

(72) Inventors: Yilong Ma, Hefei City (CN); Zhaojun Wei, Hefei City (CN); Zhi Li, Hefei City (CN); Yafang Shang, Hefei City (CN); Jianguo Zhang, Hefei City (CN); Fei Hu, Hefei City (CN); Qiya Zhang, Hefei City (CN)

(21) Appl. No.: 19/001,298

(22) Filed: Dec. 24, 2024

(30) Foreign Application Priority Data
Apr. 30, 2024 (CN) 202410539348.1

(51) Int. Cl. C07C 67/58; A61K 31/235; A61K 36/287; A61P 1/16

(52) U.S. Cl. CPC C07C 67/58; A61K 31/235; A61K 36/287; A61P 1/16; A61K 2236/55; C07C 2601/14; C07C 2601/16

(57) ABSTRACT

A method for extracting 3,5-O-dicaffeoylquinic acid from Huangshan Gongju and its application in drugs for protecting against alcoholic liver damage. The 3,5-O-dicaffeoylquinic acid has the following structural formula:

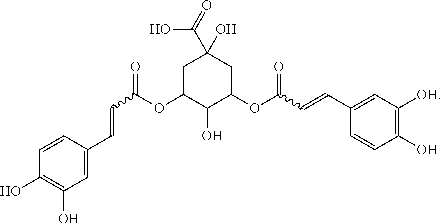 [SMILES: O=C(C=Cc1ccc(O)c(O)c1)OC1CC(O)(C(=O)O)CC(OC(=O)C=Cc2ccc(O)c(O)c2)C1O]

The present disclosure selects the medicinal and edible homology plant, Huangshan Gongju, as the research object. This plant is abundant in resources and the raw materials are easy to obtain. Specifically, the present disclosure uses 70% ethanol to extract Huangshan Gongju, uses the method of bioactivity-guided isolation, and uses a FLA-2025-10-120A preparative liquid chromatograph to isolate a phenolic compound from the active site of Gongju that resists alcoholic liver damage. The compound separation method is simple, quick, and easy to operate.

### CROSS REFERENCE TO RELATED APPLICATIONS

The present application claims the priority of Chinese patent application No. 202410539348.1, filed on Nov. 30, 2023, and the entire contents of which are hereby incorporated by reference in its entirety.

### TECHNICAL FIELD

The present disclosure relates to the field of pharmaceutical technologies, and specifically to a method for extracting 3,5-O-dicaffeoylquinic acid from Huangshan Gongju and its application in drugs for protecting against alcoholic liver damage.

### BACKGROUND

Gongju (Dendranthema morifolium (Ramat) Tzvel. cv. Gongju), also known as Huangshan Gongju or Huiju, is a well-known chrysanthemum variety in China. It is native to Shexian County and has a long history. It is a traditional medicinal and edible plant resource and has been included in the “Top Ten Anhui Medicines”. Huangshan Gongju has a short planting cycle, relatively quick returns, and extremely high benefits. Gongju is mainly distributed in the Huangshan area at higher altitudes, where the special growing environment gives it excellent quality, such that it can be used in health foods or brewed into chrysanthemum wine. Gongju is rich in chemical components such as flavonoids, phenolic acids, polysaccharides, terpenes, and essential oils. It has various active functions such as anti-oxidation, anti-tumor, antibacterial and anti-inflammatory, immune regulation, anti-epilepsy, protection of cardiovascular and liver, etc., and has broad application prospects in functional tea drinks, biomedicine, and other fields.

Huangshan Gongju contains a large portion of phenolic acids, among which 3,5-O-caffeoylquinic acid (also known as 3,5-O-dicaffeoylquinic acid) is an isochlorogenic acid formed by the esterification of quinic acid and caffeic acid. This type of compound is found in many plants and vegetables such as purple sweet potatoes, tomatoes, and Sichuan peppercorns, and has pharmacological effects such as anti-oxidation, anti-inflammation, and antibacterial properties. Studies have shown that 3,5-O-dicaffeoylquinic acid has certain antioxidant effects, inhibits α-glucosidase, and can mitigate focal cerebral ischemia-reperfusion injury in rats.

Chronic excessive alcohol-drinking is one of the main causes of liver damage in the human body. Most people who regularly drink alcohol more than 40 to 60 g/day will develop liver fat degeneration, and about 10% to 15% of these drinkers will develop cirrhosis. Alcoholic liver damage is divided into alcoholic hepatitis and alcoholic fatty liver, which can worsen into fatty hepatitis, leading to liver fibrosis, cirrhosis, and liver cancer. Excessive alcohol-drinking can lead to alcoholic liver disease through glutathione depletion, abnormal methionine metabolism, and oxidative stress which is the main cause of alcoholic liver damage. Alcohol is metabolized in the liver to produce reactive oxygen species (ROS). The accumulation of ROS in the body can lead to the peroxidation of proteins, nucleic acids, and lipids in the liver cell membrane, thereby damaging liver cells. At present, various drugs such as methaqualone, silymarin, glucocorticoids, pentoxifylline, and polyene phosphatidylcholine are applied to accelerate the removal of alcohol from the blood serum, which exert an anti-inflammatory effect to the same extent, thereby mitigating the symptoms of alcohol poisoning and preventing further deterioration of alcoholic liver damage. In recent years, it has become increasingly common to apply natural plants or their active ingredients to treat liver damage.

### SUMMARY OF THE DISCLOSURE

The purpose of the present disclosure is to provide a method for extracting 3,5-O-dicaffeoylquinic acid from Huangshan Gongju and its application in drugs for protecting against alcoholic liver damage.

To achieve the above and other related purposes, the technical solution provided by the present disclosure is: a method for extracting 3,5-O-dicaffeoylquinic acid from Huangshan Gongju, including:
S1: crushing dried Gongju, and obtaining a Gongju extract liquid by ultrasonic assistance at 50-70° C. with ethanol as an extracting agent;
S2: concentrating and drying the Gongju extract liquid to obtain a dry Gongju extract; and
S3: using a preparative liquid chromatograph, where a mobile phase is a mixed system of solvent A and solvent B, with the solvent A being methanol and the solvent B being 0.1% formic acid in water; wherein the elution gradient is as follows: 0-40 minutes, the solvent A increases linearly from 30% to 70% by volume; 40-45 minutes, the solvent A increases linearly from 70% to 100% by volume; 45-50 minutes, the solvent A decreases linearly from 100% to 30% by volume; 50-55 minutes, the solvent A is maintained at 30% by volume; wherein a flow rate of the mobile phase is 10 mL/min; and obtaining the 3,5-O-dicaffeoylquinic acid (which is also denoted as peak 2 (P2) according to the collecting order of the components).

In some embodiments, a column of the preparative liquid chromatograph is a C18 column.

To achieve the above and other related purposes, the technical solution provided by the present disclosure is: an application of the 3,5-O-dicaffeoylquinic acid in drugs for protecting against alcoholic liver damage.

In some embodiments, a structural formula of the 3,5-O-dicaffeoylquinic acid is as follows:

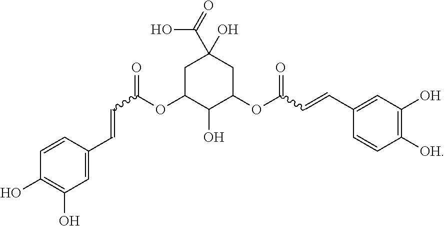 [SMILES: O=C(C=Cc1ccc(O)c(O)c1)OC1CC(O)(C(=O)O)CC(OC(=O)C=Cc2ccc(O)c(O)c2)C1O]

To achieve the above and other related purposes, the technical solution provided by the present disclosure is: a pharmaceutical composition for protecting against alcoholic liver damage, including: the 3,5-O-dicaffeoylquinic acid and a pharmaceutically acceptable excipient; wherein a dosage form of the pharmaceutical composition is a tablet, capsule, granule, pill, oral liquid, or suspension.

Due to the use of the above technical solutions, the advantages of the present disclosure over the prior art are that: The present disclosure selects the medicinal and edible homology plant, Huangshan Gongju, as the research object. This plant is abundant in resources and the raw materials are easy to obtain. Specifically, the present disclosure uses 70% ethanol to extract Huangshan Gongju, uses the method of bioactivity-guided isolation, and uses a FLA-2025-10-120A preparative liquid chromatograph to isolate a phenolic compound from the active site of Gongju that resists alcoholic liver damage. The compound separation method is simple, quick, and easy to operate.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the purity of 3,5-O-dicaffeoylquinic acid prepared according to formula I of the present disclosure.

FIG. 2 shows the mass spectrum of 3,5-O-dicaffeoylquinic acid according to formula I of the present disclosure.

FIG. 3A shows a model of cell alcohol damage; FIG. 3B shows the toxic effect of 3,5-O-dicaffeoylquinic acid on cells; and FIG. 3C shows the ameliorative effect of 3,5-O-dicaffeoylquinic acid on cell alcohol damage.

FIG. 4 shows the effect of 3,5-O-dicaffeoylquinic acid at different concentrations on the fluorescence intensity of ROS in alcohol-induced AML-12 cells.

FIG. 5 shows the effect of 3,5-O-dicaffeoylquinic acid at different concentrations on the flow cytometry results of ROS in alcohol-induced AML-12 cells.

FIGS. 6A-6D show the effect of 3,5-O-dicaffeoylquinic acid at different concentrations on various indicators (typical molecules) in alcohol-induced AML-12 cells.

FIG. 7 shows the effect of 3,5-O-dicaffeoylquinic acid at different concentrations on the fluorescence intensity of apoptosis and necrosis in alcohol-induced AML-12 cells.

FIG. 8 shows the effect of different concentrations of 3,5-O-dicaffeoylquinic acid on the flow cytometry results of apoptosis and necrosis in alcohol-induced AML-12 cells.

### DETAILED DESCRIPTION

The following specific embodiments illustrate the implementation of the present disclosure. Those skilled in the art can easily understand other advantages and effects of the present disclosure from the content disclosed in the embodiments.

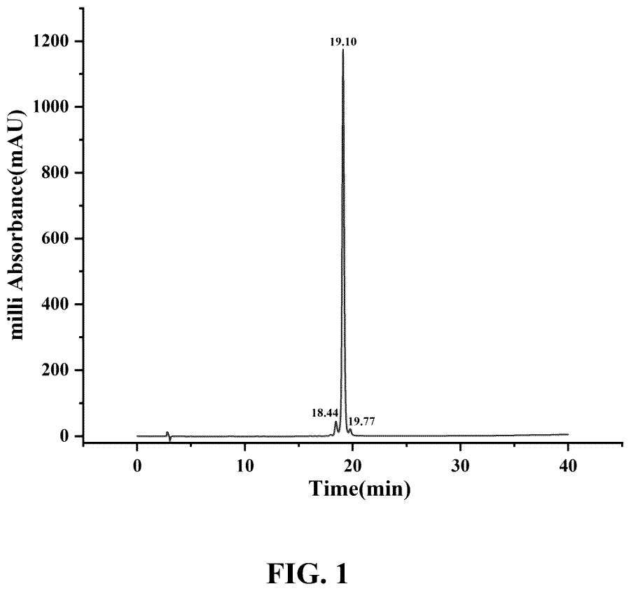

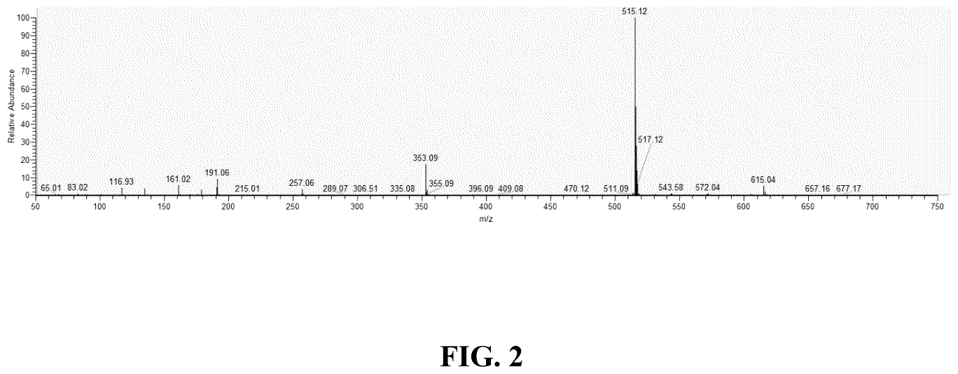

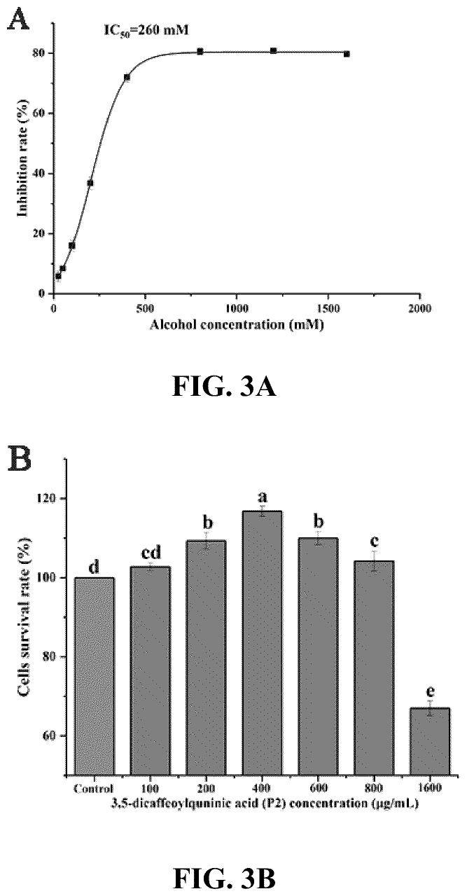

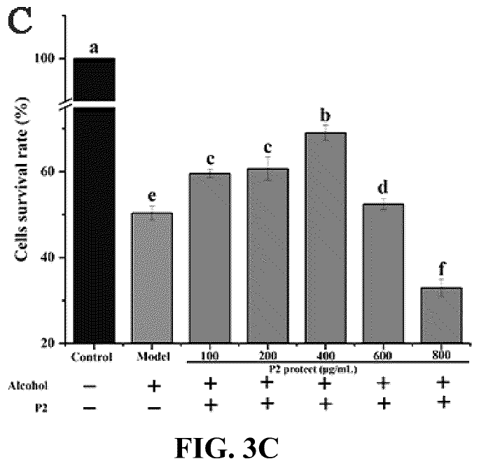

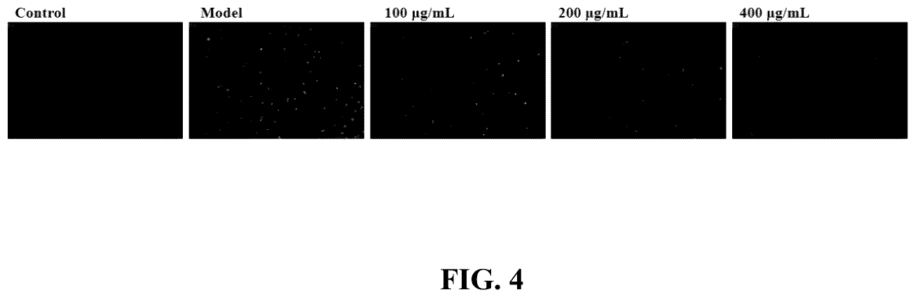

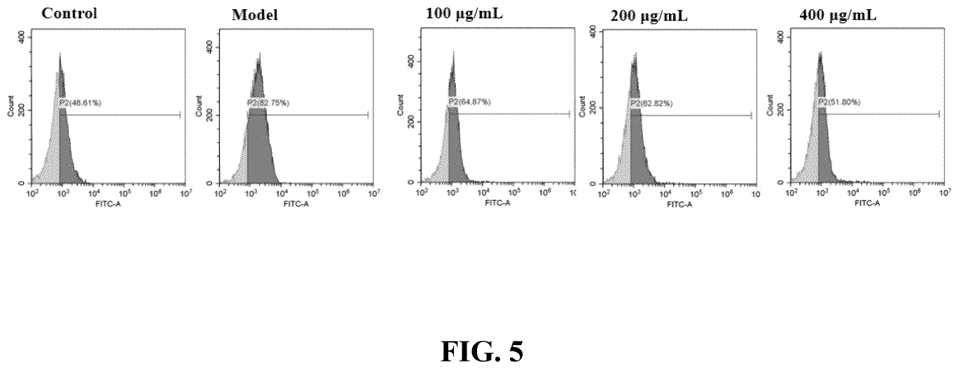

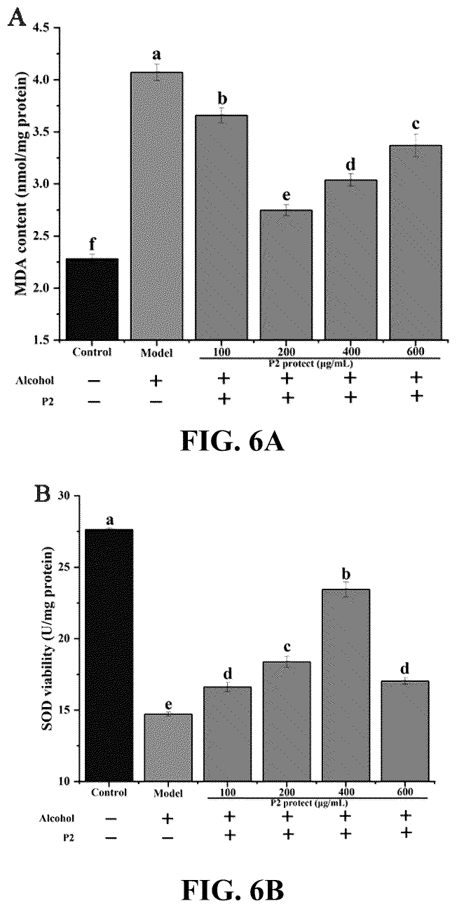

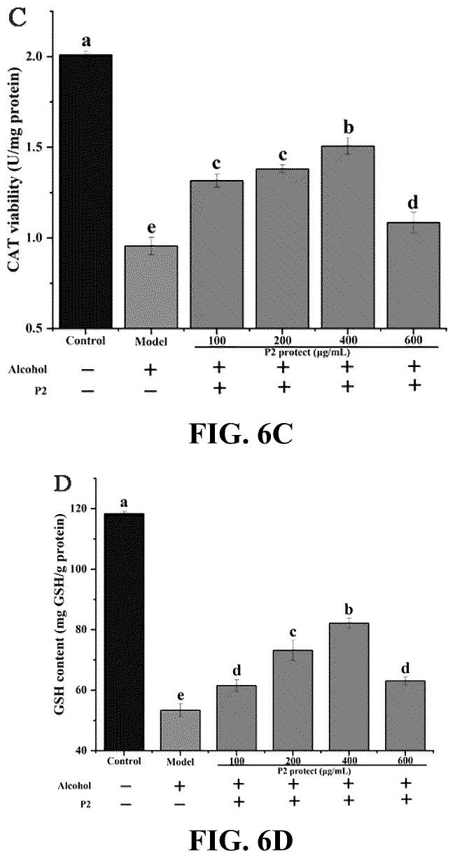

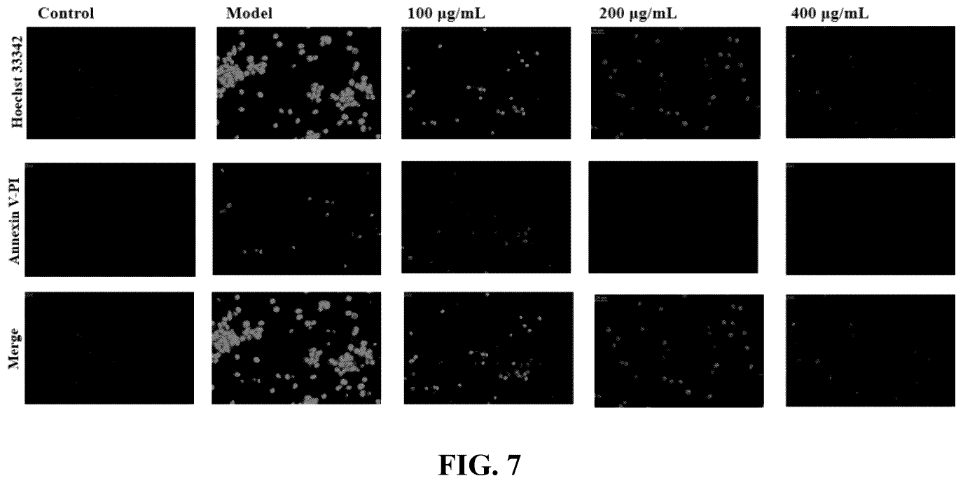

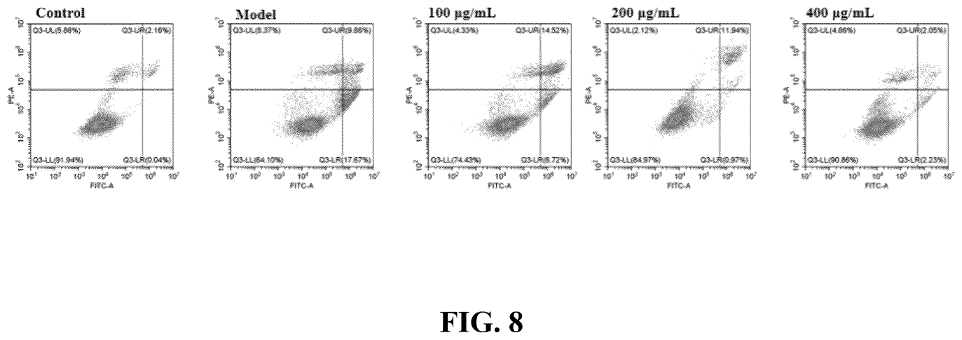

Referring to FIGS. 1-2, it should be noted that the structures, proportions, and sizes depicted in the drawings are only intended to illustrate the contents of the specification, for understanding and reading by those skilled in the art, and are not intended to limit the conditions under which the present disclosure may be practiced. Therefore, they are not of technical significance, and any modifications to the structures, changes in proportional relationships, or adjustments of size are not intended to be limited by the present disclosure. The following embodiments are provided for a better understanding of the present disclosure and are not intended to limit the present disclosure. Unless otherwise specified, the experimental methods in the following embodiments are routine methods. The experimental materials used in the following embodiments are commercially available from regular biochemical reagent stores, unless otherwise specified.

### Embodiment 1: A Method for Extracting 3,5-O-Dicaffeoylquinic Acid from Huangshan Gongju and its Application in Drugs for Protecting Against Alcoholic Liver Damage

A 3,5-O-dicaffeoylquinic acid (3,5-O-dicaffeoylquinic acid) with a structure as shown in Formula I is isolated from Huangshan Gongju:

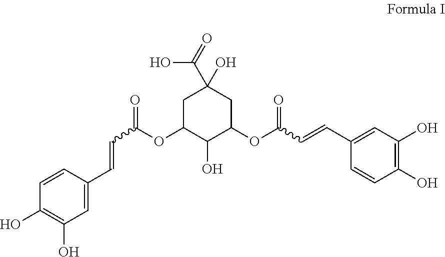 [SMILES: O=C(C=Cc1ccc(O)c(O)c1)OC1CC(O)(C(=O)O)CC(OC(=O)C=Cc2ccc(O)c(O)c2)C1O]

A method for preparation and identification of 3,5-O-dicaffeoylquinic acid, including the following steps:

(1): dried Gongju is crushed, and Gongju extract liquid is obtained by assisted extraction with 70% ethanol (solid-liquid ratio 1:20) at 60° C. for 1 h with ultrasonic extraction.

(2): the Gongju extract liquid is concentrated under vacuum at 50-55° C. and dried (using a freeze dryer) to obtain a dry Gongju extract.

(3): using a preparative liquid chromatograph (FLA-2025-10-120A) equipped with a 5 μm×4.6 mm×250 mm C18 column, a sample (10 mL) is eluted with methanol (solvent A) and 0.1% formic acid in water (solvent B) as follows: 0-40 min, solvent A linearly increased from 30% to 70% by volume; 40-45 min, solvent A linearly increased from 70% to 100% by volume; 45-50 min, solvent A linearly decreased from 100% linearly decreased to 30%; 50-55 minutes, solvent A is maintained at 30% by volume, of which the flow rate is 10 mL/min, and 3,5-O-dicaffeoylquinic acid is prepared. Preparation method of 0.1% formic acid water: 1 mL formic acid is added to 1 L ultrapure water, mixed well, filtered through a 0.22 μm filter membrane, and the filtrate is sonicated for 10 min and then stored.

(4): the phenolic compounds are analyzed using high-performance liquid chromatography (iChorm5100, Elite, Dalian, China) with a 20×250 mm-10 μm-C18 column and identified at 350 nm. A sample (20 μL) is eluted with methanol (solvent A) and 0.1% formic acid in water (solvent B). The elution conditions are as follows: 0-10 minutes, solvent A linearly increased from 15% to 40%; 10-40 minutes, solvent A linearly increased from 40% to 50%; 40-45 minutes, solvent A linearly increased from 50% to 100%; 45-50 minutes, solvent A linearly decreased from 100% to 15%; 50-55 minutes, solvent A maintained at 15%, where the flow rate is 1 mL/min. The LC-MS is equipped with a ZORBAX Eclipse Plus C18 column. Mass spectrometry is measured using negative electrospray ionization in the m/z range of 100-1000. The ionization voltage of the sample is 3.5 kV, and the sample flow rate is 11 L/mL. The capillary temperature is 350° C., the capillary voltage is 4000V (+)/3500V (−), and the nebulizer pressure is 50 psi.

(5): comparison with the standard substance for determining that the analyzed sample is 3,5-O-dicaffeoylquinic acid.

The cell survival rate, ROS, MDA, SOD, CAT, and GSH content in normal mouse liver cells are detected using biochemical methods to screen and evaluate the protective effect of 3,5-O-dicaffeoylquinic acid against alcoholic liver damage. It is found that this compound has a certain protective effect against alcoholic liver damage. Therefore, another purpose of the present disclosure is to provide an application of 3,5-O-dicaffeoylquinic acid in the preparation of drugs for protection of alcoholic liver damage.

The dosage form of the drug is any dosage form approved in pharmacy; in some embodiments, the dosage form is a tablet, capsule, granule, pill, oral liquid, or suspension.

FIG. 1 shows the purity of 3,5-O-dicaffeoylquinic acid as shown in Formula I of the present disclosure.

The compound of Formula I is a white solid powder, and high-performance liquid chromatography (HPLC) results show that the main peak of the compound after extraction and purification appears at 19.10 min (FIG. 1). The [M-H]− peak at m/z 515.12 indicates that the compound has a relative molecular mass of 516.12. Meanwhile, the compound is further identified as 3,5-O-dicaffeoylquinic acid with the molecular formula C₂₅H₂₄O₁₂ by comparing it with the standard product using HPLC.

Investigation of the protective effect of compound I against alcoholic liver damage:

### 1. Experimental Cells, Reagents, and Instruments

AML-12 cells, mouse normal liver cells; 3-(4,5-dimethylthiazol-2-yl)-2,5-diphenyl-2H-tetrazolium-3-bromide (MTT), dimethyl sulfoxide (DMSO), reactive oxygen species (ROS) kit, superoxide dismutase (SOD) kit, malondialdehyde (MDA) kit, glutathione (GSH) kit; high-speed centrifuge, microplate reader, etc.

### 2. Model Establishing, and Screening for Suitable Drug (Meaning 3,5-O-Dicaffeoylquinic Acid in the Following Text) Concentrations

Logarithmically growing AML-12 cells are seeded into 96-well plates at a density of 0.8×10⁵ cells/mL, and 100 μL of cell suspension is added to each well and incubated for 24 hours. In an alcohol treatment group, alcohol (0-1600 mM, DMEM dilution) is added to the incubator for 24 hours. In a 3,5-O-dicaffeoylquinic acid treatment group, 100 μL of 3,5-O-dicaffeoylquinic acid (100-1600 μg/mL, DMEM dilution) is added to the incubator for 24 hours. 120 μL of MTT (1 mg/mL) is added to each well and incubated for 4 h. The reaction solution is discarded; 150 μL DMSO is added, and OD value is measured at a wavelength of 490 nm after shaking for 10 minutes in the dark.

### 3. Administration

Log-growing AML-12 cells are collected and digested, and then seeded into a 96-well plate at a density of 0.8×10⁵ cells/mL per well. After 24 hours of cell proliferation, the control (and model) group is incubated with 100 μL of medium for 48 hours; the 3,5-O-dicaffeoylquinic acid treatment group is incubated with different concentrations of 3,5-O-dicaffeoylquinic acid for 24 hours, and the original medium is discarded; the cells (in model and drug treated group) are incubated in 100 μL of medium containing 260 mM alcohol for 24 hours. The absorbance measurement method is the same as the item 2 above.

Cell survival rate is calculated using the following formula:

$$cell survival rate ( \% ) = \frac{{OD}_{experimental group} - {OD}_{blank group}}{{OD}_{comparative group} - {OD}_{blank group}}$$

### 4. ROS Content Determination

AML-12 cells are treated with drugs (100, 200, 400 μg/mL) for 24 h, and then treated with 260 mM alcohol for 24 h. After trypsin digestion and PBS washing, the probe from the ROS kit is added. First, incubation is conducted with 1 mL of DCFH-DA (diluted in serum-free DMEM) at 37° C. for 20 min. The supernatant is discarded after centrifugation, and the remaining is washed three times and resuspended in PBS. 10 μL of the resuspension is observed under a fluorescence microscope, and is further detected under a flow cytometer.

### 5. Determination of MDA, SOD, CAT, and GSH Content

Excessive ROS in cells can cause oxidative reactions in proteins and unsaturated fatty acids. MDA is the final product of the oxidation of polyunsaturated fatty acids and is a marker of lipid peroxidation in cells, which is widely used in studies on oxidative stress. SOD, CAT, and GSH are important antioxidant enzymes in the body and play an important role in the body's systematic resistance to oxidative stress. Based on above, AML-12 cells are treated with drugs (100, 200, 400 μg/mL) for 24 h, and then treated with 260 mM alcohol for 24 h. After trypsin digestion and PBS washing, the MDA, SOD, CAT, and GSH content in the cells are detected according to the guidance of corresponding kits.

6. Ameliorative Effect of 3,5-O-Dicaffeoylquinic Acid on Apoptosis in Cells after Alcohol Damage

AML-12 cells are treated with drugs (100, 200, 400 μg/mL) for 24 h, and then treated with 260 mM alcohol for 24 h. After trypsin digestion and PBS washing, probe from an apoptosis detection kit is added. First, incubation is conducted with 5 μL Hoechst (diluted in PBS) at 0° C. for 20 min. The supernatant is discarded after centrifugation, and the remaining is washed three times and resuspended in PBS. 10 μL of the resuspension is observed under a fluorescence microscope, and is further detected under a flow cytometer.

### 7. Experimental Results

### 7.1. Toxicity of 3,5-O-Dicaffeoylquinic Acid and Protective Effect on Alcohol-Damaged AML-12 Cells

The experimental results show that: after screening the oxidative damage effect of alcohol on AML-12 cells at concentrations of 0-1600 mM, the survival rate of cells decreased by 50% when treated with 260 mM alcohol, and this concentration can be used for subsequent research. This experiment studies the cell survival rate of AML-12 treated with 3,5-O-dicaffeoylquinic acid (100-1600 μg/mL) in order to find a concentration range with no significant toxic effect on the cells. The results show that the cell survival rate decreases significantly (P<0.05) when the concentration of 3,5-O-dicaffeoylquinic acid is 1600 μg/mL, whereas the cell survival rate increases significantly when the concentration of 3,5-O-dicaffeoylquinic acid is 0-800 μg/mL, indicating that 3,5-O-dicaffeoylquinic acid is non-toxic to AML-12 cells in the concentration range of 0-800 μg/mL, and is therefore selected for further research. In addition, compared with a model group, 3,5-O-dicaffeoylquinic acid at a concentration of 100-400 g/mL can significantly increase the survival rate of alcohol-damaged AML-12 cells, and the concentration of 400 μg/mL has the highest cell survival rate, which rate is significantly increased by 19.90% compared with the model group, indicating that 3,5-O-dicaffeoylquinic acid can protect AML-12 cells and reduce the damaging effect of alcohol on cells.

### 7.2. Influence of 3,5-O-Dicaffeoylquinic Acid on the ROS Fluorescence Intensity (and Flow Cytometry Results) of Ethanol-Induced AML-12 Cells

The results show that pretreatment with 3,5-O-dicaffeoylquinic acid at a concentration of 400 μg/mL significantly reduces the ROS fluorescence intensity of the cells compared to the alcohol-treated group (model group). Fluorescence intensity increases with the ROS level. The fluorescence intensity of the model group treated with alcohol is significantly higher than that of the control group, while the fluorescence intensity of ROS in the group treated with 3,5-O-dicaffeoylquinic acid is weaker than that of the model group, and it gradually weakens with the increase of drug concentration. The results of flow cytometry show that, compared with the control group, after alcohol damage, the peak fluorescence intensity of the model group shifts significantly to the left, and the percentage of ROS-positive cells increases by 34.15%, indicating that alcohol can stimulate cells to produce ROS and cause damage. After pretreatment with drugs of different concentrations, as the drug concentration increases, the peak fluorescence intensity shifts significantly to the right, indicating that the drug can reduce the damaging effect of alcohol on cells caused by ROS.

### 7.3 Influence of 3,5-O-Dicaffeoylquinic Acid on the Levels of MDA, SOD, CAT, and GSH in Ethanol-Induced AML-12 Cells

The results show that alcohol can affect the intracellular antioxidant system, resulting in a significant increase in MDA levels and a significant decrease in SOD and CAT activity and GSH levels. After pretreatment with 3,5-O-dicaffeoylquinic acid at different concentrations, the MDA content in cells significantly reduces, with the most significant effect at a concentration of 200 μg/mL. Furthermore, concentrations of 100-600 μg/mL can significantly restore the activity of SOD and CAT in cells, which mitigates the oxidative damage to cells. In addition, after pretreatment with 3,5-O-dicaffeoylquinic acid (400 μg/mL), the GSH content increases from 53.39±2.16 mg/g protein to 82.14±1.67 mg/g protein.

### 7.4 Influence of 3,5-O-Dicaffeoylquinic Acid on Ethanol-Induced Apoptosis in AML-12 Cells

The results show that pretreatment with 3,5-O-dicaffeoylquinic acid at a concentration of 400 μg/mL significantly reduces the fluorescence intensity of apoptosis compared with the alcohol-treated group. The fluorescence intensity increases with the increase of apoptosis. The fluorescence intensity of the alcohol-treated model group is significantly higher than that of the control group, while the fluorescence intensity of apoptosis after 3,5-O-dicaffeoylquinic acid pretreatment is weaker than that of the model group. Apoptosis results show that in the control group, the cell survival rate is as high as 91.94%; after alcoholic damage, the cell survival rate decreases significantly, and the ratio of apoptosis to necrosis increases significantly. After pretreatment with drug of different concentrations, the cell survival rate gradually increases with the increase in drug concentration, indicating that the drug can improve the alcoholic damage to cells.

The above is only intended to explain some embodiments of the present disclosure, and it is not intended to limit the present disclosure in any way. Therefore, any modifications or changes related to the present disclosure made in the same inventive spirit should still be included in the scope of the present disclosure.

What is claimed is:

1. A method for extracting 3,5-O-dicaffeoylquinic acid from Huangshan Gongju, comprising:
S1: crushing dried Gongju, and obtaining a Gongju extract liquid by ultrasonic assistance at 50-70° C. with ethanol as an extracting agent;
S2: concentrating and drying the Gongju extract liquid to obtain a dry Gongju extract; and
S3: using a preparative liquid chromatograph, where a mobile phase is a mixed system of solvent A and solvent B, with the solvent A being methanol and the solvent B being 0.1% formic acid in water; wherein the elution gradient is as follows: 0-40 minutes, the solvent A increases linearly from 30% to 70% by volume; 40-45 minutes, the solvent A increases linearly from 70% to 100% by volume; 45-50 minutes, the solvent A decreases linearly from 100% to 30% by volume; 50-55 minutes, the solvent A is maintained at 30% by volume; wherein a flow rate of the mobile phase is 10 mL/min; and obtaining the 3,5-O-dicaffeoylquinic acid.

2. The method according to claim 1, wherein a column of the preparative liquid chromatograph is a C18 column.

3. A method for protecting against alcoholic liver damage, comprising:
applying a drug comprising the 3,5-O-dicaffeoylquinic acid prepared by the method according to claim 1.

4. The application according to claim 3, wherein a structural formula of the 3,5-O-dicaffeoylquinic acid is as follows:

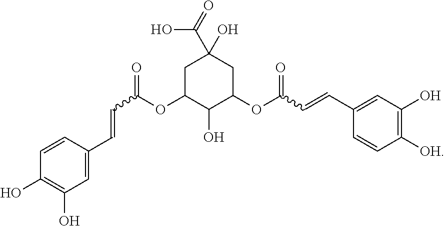 [SMILES: O=C(C=Cc1ccc(O)c(O)c1)OC1CC(O)(C(=O)O)CC(OC(=O)C=Cc2ccc(O)c(O)c2)C1O]

5. A pharmaceutical composition for protecting against alcoholic liver damage, comprising: the 3,5-O-dicaffeoylquinic acid prepared by the method according to claim 1, and a pharmaceutically acceptable excipient; wherein a dosage form of the pharmaceutical composition is a tablet, capsule, granule, pill, oral liquid, or suspension.
